(19) United States
(12) Patent Application Publication
(10) Pub. No.: US 2025/0331982 A1
(43) Pub. Date: Oct. 30, 2025

(54) IMPLANT DELIVERY DEVICE

(71) Applicant: EMBODY, INC., Norfolk, VA (US)

(72) Inventor: Stacy Blair, Norfolk, VA (US)

(21) Appl. No.: 19/184,382

(22) Filed: Apr. 21, 2025

Related U.S. Application Data
(60) Provisional application No. 63/639,334, filed on Apr. 26, 2024.

(51) Int. Cl. A61F 2/08

(52) U.S. Cl. CPC A61F 2/0805

(57) ABSTRACT

The present application discusses and illustrates various examples including implant delivery device. This can optionally include handle, a first shaft and an implant holder. The handle can be disposed at a proximal end of the implant delivery device and configured to be grasped by a user. The first shaft can extend distally from the handle. The implant holder can be coupled to the first shaft. The implant holder can be configured to retain a sheet-like implant adjacent two or more corners of the sheet-like implant during implantation of the sheet-like implant into a patient at a surgical site.

### CLAIM OF PRIORITY

This application claims the benefit of U.S. Provisional Patent Application Ser. No. 63/639,334, filed on Apr. 26, 2024, the benefit of priority of which is claimed hereby, and which is incorporated by reference herein in its entirety.

### BACKGROUND

The present embodiments relate generally to medical devices, and in particular to medical devices used to repair tissue.

Rotator cuff repair is a surgical procedure performed to repair torn (or partially torn) tendons in the shoulder. This procedure can be done with large incisions or with arthroscopic techniques. To repair a torn tendon (such as the supraspinatus tendon), a surgeon may use anchors and sutures to reattach the tendon to the humerus bone. The repaired area may then be covered with a graft to facilitate healing. Inserting a graft through a small incision and laying it down in a desired position can be difficult with arthroscopic surgery.

There is a need in the art for a system and method that addresses the shortcomings discussed above.

### OVERVIEW

In one aspect, the present disclosure is directed to an implant delivery device optionally including a handle disposed at a proximal end of the implant delivery device and configured to be grasped by a user, a first shaft extending distally from the handle and an implant holder coupled to the first shaft, the implant holder being configured to retain a sheet-like implant adjacent two or more corners of the sheet-like implant during implantation of the sheet-like implant into a patient at a surgical site.

Implant delivery devices including those used to deliver a sheet-like implant such as Tapestry® Biointegrative Implant manufactured by Zimmer Biomet Inc. of Warsaw, IN are known. Examples of such devices include application Ser. No. 18/226,560 (U.S. Patent Application Pub. No. 2024/0033105), the entire disclosure of which is incorporated herein by reference. The implant delivery device and the methods disclosed herein provide improvements including enhanced stability and positionability, improved implant engagement with and disengagement from the device, an open frame design having windows for suture anchor placement and improved capture and retention of the implant among other benefits. For example, the delivery device can include an implant holder that holds the implant in a desired position (e.g., a substantially flat position) for delivery to tissue that is being repaired. This reduces the need for manipulation by the surgeon to place the implant in a desired orientation for receiving suture anchor(s), for example. Additionally, the implant holder provides stability by supporting the implant adjacent or at one or more corners and/or adjacent or at edges of the implant. Furthermore, the implant holder engages adjacent or at one or more corners and/or adjacent or at edges of the implant, which not only maintains the implant in the desired delivery position (e.g., the substantially flat position) but also allows for the windows (open areas in the middle of the implant) to be exposed to receive the suture anchor(s). The implant holder can retain/hold the using one or more slots, these slots can be positioned to receive the implant adjacent one or more corners thereof. Use of the one or more slots allows the implant to be disengaged from the implant holder upon proximal withdrawal of the implant holder. This feature improves placement of the implant while the delivery device is removed, minimizing the risk of undesired displacement and/or tearing of the implant. The one or more slots additionally are configured to reduce or prevent tearing of fibers of the implant fibers and can be configured to modulate a force of pinching by the implant holder on the implant. Thus, the one or more slots facilitate that the implant is held securely without damaging its structural integrity.

Other devices, systems, methods, features, and advantages of the embodiments will be, or will become, apparent to one of ordinary skill in the art upon examination of the following figures and detailed description. It is intended that all such additional systems, methods, features, and advantages be included within this description and this summary, be within the scope of the embodiments, and be protected by the following claims.

### BRIEF DESCRIPTION OF THE DRAWINGS

The embodiments can be better understood with reference to the following drawings and description. The components in the figures are not necessarily to scale, emphasis instead being placed upon illustrating the principles of the embodiments. Moreover, in the figures, like reference numerals designate corresponding parts throughout the different views.

FIG. 1 is a schematic view of a step in a procedure for repairing a rotator cuff tendon according to an embodiment;

FIG. 2 is a schematic view of a step of applying an implant to a portion of a rotator cuff tendon to facilitate healing according to an embodiment;

FIG. 3 is a schematic illustration of an implant delivery device according to an embodiment holding an implant prior to delivery through an illustrated access cannula;

FIG. 4 is an enlarged schematic view of a distal end of the implant delivery device including an implant holder holding an implant;

FIG. 5A is a plan view of a first shaft and the implant holder of FIGS. 3 and 4 retaining the implant;

FIG. 5B is a plan view of the first shaft and the implant holder of FIG. 5A with the implant removed;

FIG. 6 is a plan view of another example of an implant holder retaining the implant;

FIG. 7 is a plan view of yet another example of an implant holder retaining the implant;

FIG. 8 is a plan view of yet another example of an implant holder without the implant;

FIG. 9 is a schematic view of an implant delivery device according to an embodiment holding an implant prior to delivery through an illustrated access cannula;

FIGS. 10-11 are schematic views showing the implant being inserted through the cannula with the implant being rolled up as it is during delivery through the cannula;

FIG. 12 is a schematic view of the implant delivery device extending through the cannula with the cannula sealing member seated within the cannula and with the implant unfurled to a delivery position after exiting the cannula;

FIG. 13 is a schematic view of the implant delivery device with second shaft the supports the implant partially withdrawn during the process of releasing the implant;

FIG. 14 is an enlarged schematic view of the second shaft of the implant delivery device entirely withdrawn;

FIG. 15 is a schematic view of the implant holder of the implant delivery device with the implant supporting flange member fully withdrawn and with the implant released from the implant delivery device;

FIG. 16 is a flowchart illustrating a method of delivering a sheet-like implant to a surgical site using the implant delivery devices shown and discussed with respect to FIGS. 3-15.

### DETAILED DESCRIPTION

For clarity, the description makes reference to distal and proximal directions (or portions). As used herein, the term “distal” shall refer to a direction or portion oriented or located away from a user who is holding the implant delivery device (i.e., away from a surgeon using the device and toward a patient into which the device is being inserted). The term “proximal” shall refer to a direction or portion oriented or located toward a user who is holding the implant delivery device (i.e., toward a surgeon using the device and away from a patient into which the device is being inserted).

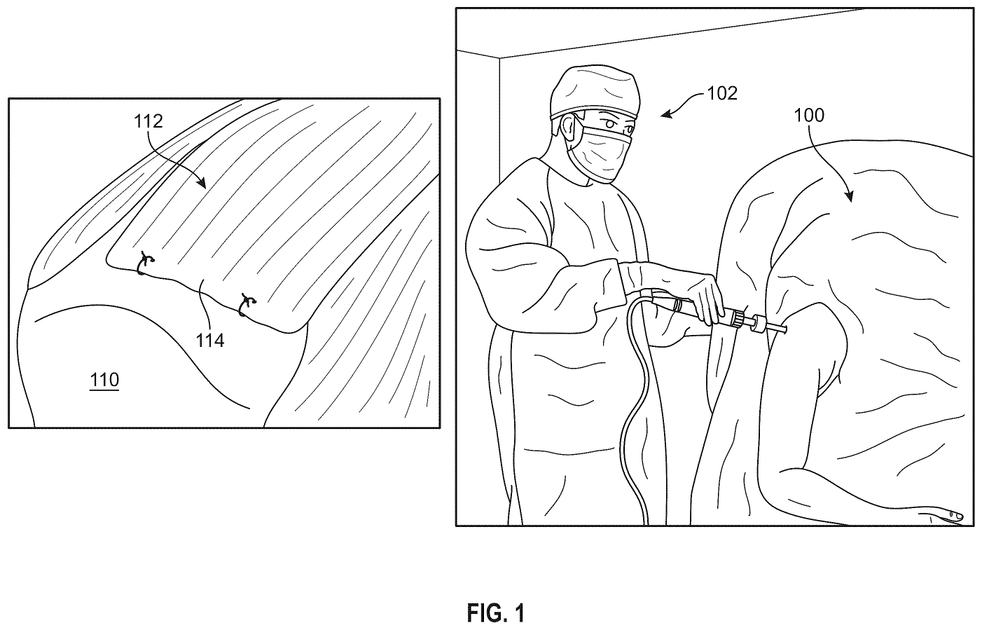

FIG. 1 is a schematic view illustrating a surgical procedure to repair a tendon in a patient's shoulder. Specifically, a patient 100 is undergoing arthroscopic surgery that is performed by surgeon 102. Also shown in FIG. 1 is an enlarged view of a portion of humerus 110 and rotator cuff tendons 112. In the present example, surgeon 102 has recently applied anchors and sutures to secure supraspinatus tendon 114 to humerus 110.

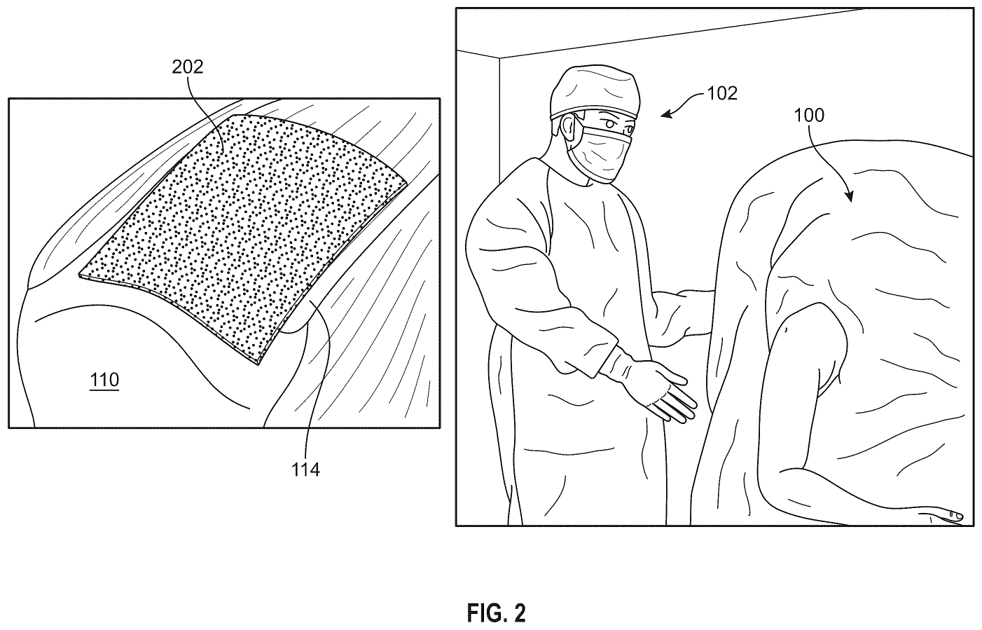

Once the tendon has been sufficiently repaired and/or the surgical site is otherwise prepared, surgeon 102 may insert an implant, such as a graft, through an incision (possibly using another device to facilitate insertion). The graft can then be placed over the tendon and/or portion of the underlying bone in order to facilitate healing. As an example, FIG. 2 shows a schematic view of a graft 202 (sometimes referred to as a sheet-like implant or simply implant elsewhere herein) that has been applied over the tendon 114 as well as over a portion of humerus 110.

Although the exemplary embodiment depicts a procedure in which a tendon is first secured to the bone using sutures and anchors, in other embodiments a graft can be applied to one or more tendons without first reattaching a tendon. For example, grafts could be applied to tendons that have only partial tears.

Once graft 202 has been placed over the tendon, one or more sutures or anchors are required to hold graft 202 in place. The present embodiments disclose a graft delivery device configured to hold an implant, such as the graft 202, during insertion and release the implant/graft once it has been secured at the surgical site.

An embodiment of an implant delivery device that can be used to insert an implant through a small incision and position it in over tissue in a fully opened delivery position.

The implant delivery device can be configured with an implant holder for biasing a sheetlike implant to an open, unfurled configuration as well as for releasing the implant once it has been placed in a desired location. The handle can be configured to actuate components such as a second shaft of the device. It will be understood that the disclosed implant delivery device may be configured to deliver any type of sheet-like implant. For example, in some embodiments, the delivery device may be used to deliver a sheet-like graft. In some embodiments, the graft may be formed, at least in part of collagen. In other embodiments, the sheet- like implant may be formed of synthetic material or blends of collagen and various synthetic polymers as are described, for example, in Francis et al., U.S. Pat. No. 10,617,787, issued Apr. 14, 2020, and entitled “Biopolymer Compositions, Scaffolds and Devices,” the entire disclosure of which is incorporated herein by reference, and Francis et al., U.S. Pat. No. 10,653,817, issued May 19, 2020, and entitled “Method for Producing an Implantable Ligament and Tendon Repair Device,” the entire disclosures of each of which is incorporated herein by reference.

application Ser. No. 18/226,560 (incorporated previously) describes one option for the handle includes a slider member coupled to components such as a second shaft (e.g., a wire) that facilitate releasing the implant once it has been placed in a desired position at a surgical site in the body of the patient.

In some embodiments, the disclosed implant delivery device can be configured for insertion through a medical cannula. Medical cannulas are generally well-known in the art of arthroscopic surgery. For example, various types of cannulas are used to control the inflow or outflow of fluids, to allow access for tools into the tissue, and for other functions. In some types of surgeries, an implant or other substrate material may be introduced into a surgical site through a cannula, which maintains an enlarged access port to the surgical site. Additionally, many arthroscopic surgeries, such as joint surgeries, use pressurized irrigation fluid to keep tissue separated apart from other tissue. In particular, pressurized irrigation fluid may be used to aid in visualization of the surgical site as well as to prevent bleeding from vasculature surrounding the surgical cuts. Other types of surgeries, such as gastrointestinal procedures, use pressurized gas to provide access to and visualization of the surgical site.

Utilizing a cannula eliminates the need to include an outer sheath covering the implant during insertion. That is, if the implant is inserted through an incision in the bare skin of the patient, the implant, which is typically relatively delicate, is typically preferred to be encased. Accordingly, implant delivery devices configured to introduce implants directly through the skin (i.e., without a cannula) typically include a sheath that covers the implant during insertion and which is then retracted to expose the implant once the distal end of the instrument reaches the surgical site. Alternatively, devices may include a fixed outer sheath and a movable inner component that holds the implant within the sheath during insertion and is then moved distally to expose the implant at the surgical site.

The presently disclosed implant delivery device is devoid of implant-covering sheathes. Instead, the disclosed device includes a deformable and resilient implant holder that retains the implant such as adjacent one or more corners thereof. The resilient implant holder thus can bias the implant toward an unfurled configuration that is desirable for implantation (e.g., a substantially flat position). However, because the implant holder can be configured as a card and can be made of a flexible material such as thermoplastic polyester elastomer such as Hytrel®, the implant (and the implant holder) may roll up when delivered through a surgical cannula. When the distal end of the device exits the distal end of the cannula, the implant holder is configured to bias the implant into the unfurled configuration. The implant can then be positioned over the tendon and/or bone at the surgical site. In addition, the implant delivery device can be used to hold the implant in place while anchors are used to secure the implant to the native tissue/bone.

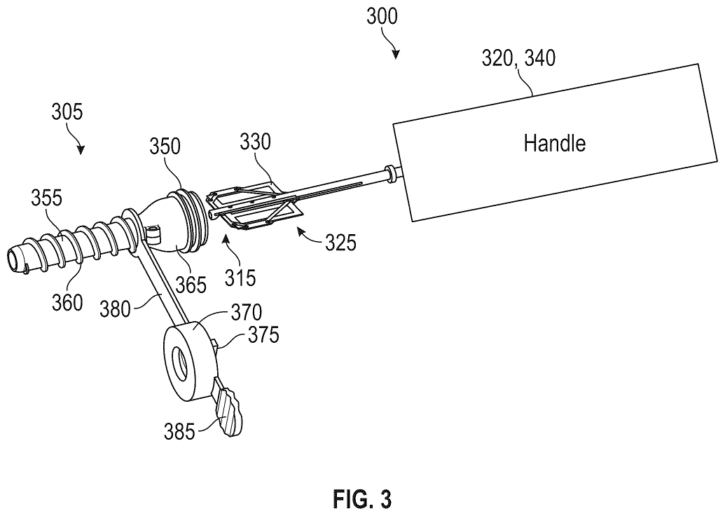

FIG. 3 is a schematic illustration of an implant delivery device according to an embodiment holding an implant prior to delivery through an illustrated access cannula. As shown in FIG. 3, a distal end portion of the implant delivery device 300 may be configured to be inserted through a cannula 305. As further shown in FIG. 3, implant delivery device 300 may have a proximal end 310 and a distal end 315. In addition, implant delivery device 300 may include a handle 320 disposed at proximal end 310 of implant delivery device 300 and configured to be grasped by a user.

Implant delivery device 300 may include an implant holder 325 at or adjacent the distal end 315 of implant delivery device 300, implant holder 325 being configured to retain a sheet-like implant 330 during implantation of implant 330.

Handle 320 can include an actuator 340 such as a slider member or trigger configured to actuate an implant release mechanism such as a second shaft as discussed in greater detail below.

As shown in FIG. 3, exemplary cannula 305 may have a proximal end 350 with a wider mouth opening and a narrower distal portion 355 including threads 360. Cannula 305 may include a tapered intermediate portion 365 between proximal end 350 and distal portion 355. Implant delivery device 300 may be configured such that, when passing implant 330 through cannula 305, tapered intermediate portion 365 may roll up implant 330. As further shown in FIG. 3, cannula 305 may include a seal structure 370. Seal structure may include a seal 375. Seal 375 may be any type of seal suitable for passing instruments, such as obturators through. For example, as shown in the accompanying figures, seal 375 may be a duckbill valve. In other embodiments, seal 375 may be a tricuspid valve. In some embodiments, seal structure 370 may include more than one seal in series. In addition, seal structure 370 may include a tether 380 for connecting seal structure 370 to cannula 305. Further, seal structure 370 may include a grasping tab 385. It will be understood that cannula 305 and seal structure 370 may have any suitable configurations for passing implants therethrough using implant delivery device 300. Implant delivery device 300 may be configured for use with cannulas having any of the features disclosed in Jones et al., U.S. patent application Ser. No. 17/173,531, filed Feb. 11, 2021, and entitled “Surgical Cannula with Removable Pressure Seal,” the entire disclosure of which is incorporated herein by reference.

Further, implant delivery device 300 may include a first shaft 390, which can be an outer shaft having a cannula, extending from handle 320 to distal end 315 of implant delivery device 300. First shaft 390 may be formed out of a substantially rigid material that is also biocompatible. For example, first shaft 390 may be formed out of surgical stainless steel or titanium. In some embodiments, first shaft 390 may be formed to have substantially no deflection when subjected to the forces of implant delivery. In other embodiments, first shaft 390 may be configured to have a predetermined amount of flexibility in order to facilitate placement of the implant via manipulation of the handle. It will be understood that, also contemplated are configurations where the implant is not necessarily attached to a shaft, but attached to an alternative structure at the distal end of the device.

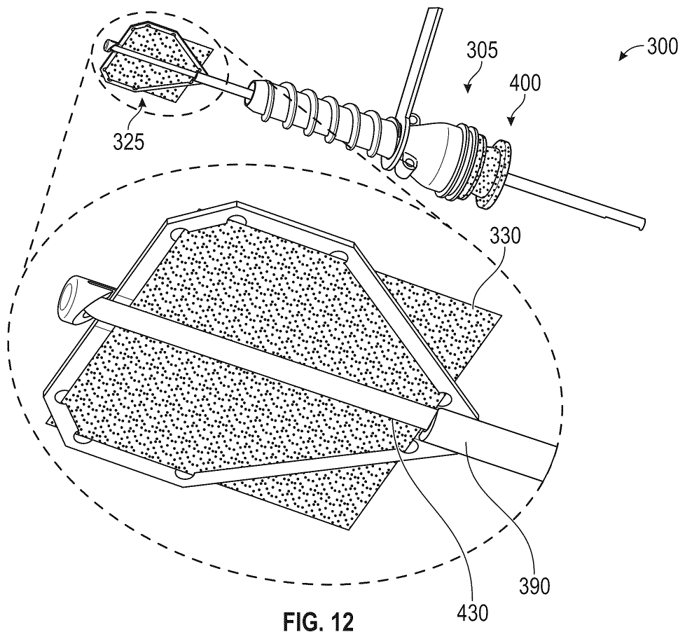

In addition, as shown in FIG. 3, implant delivery device 300 may further include a cannula sealing member 400 (illustrated in FIG. 12) disposed about a midportion of first shaft 390. Cannula sealing member may include a proximal flange for abutting proximal end 350 of cannula 305. In addition, cannula sealing member may include a narrower distal portion configured to fit within narrow distal portion 355 of cannula 305. Distal portion of cannula sealing member may include one or more ribs configured to seal against the inner wall of distal portion 355 of cannula 305. In addition, cannula sealing member may include a tapered intermediate portion between proximal flange and narrow distal portion.

When implant delivery device 300 is to be inserted through cannula 305, seal structure 370 may be removed from proximal end 350 of cannula 305. In order to maintain pressure at the surgical site, cannula sealing member may be inserted into cannula 305 as implant delivery device 300 is passed through cannula 305. Cannula sealing member may be slidable along first shaft 390 of implant delivery device 300 in order to facilitate manipulation of implant delivery device 300 to and about the surgical site.

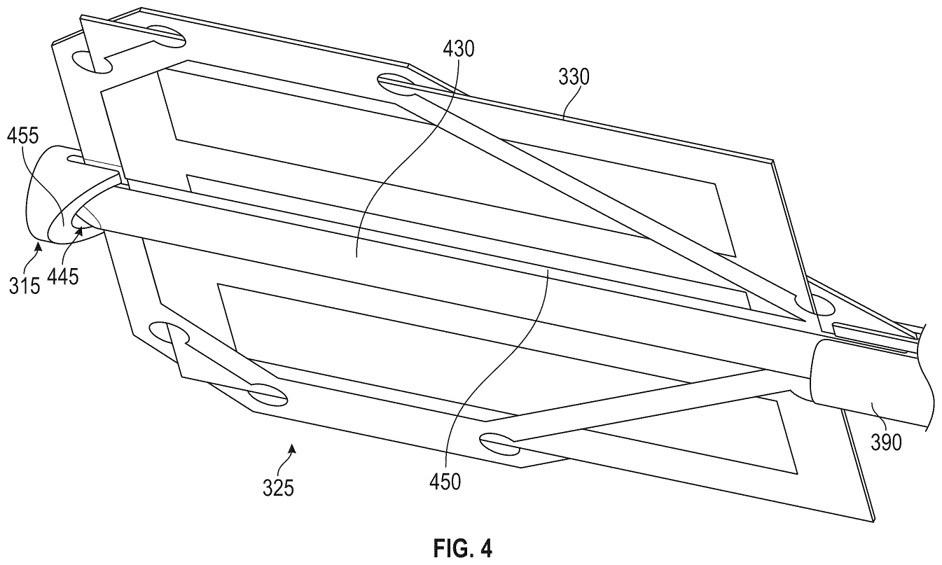

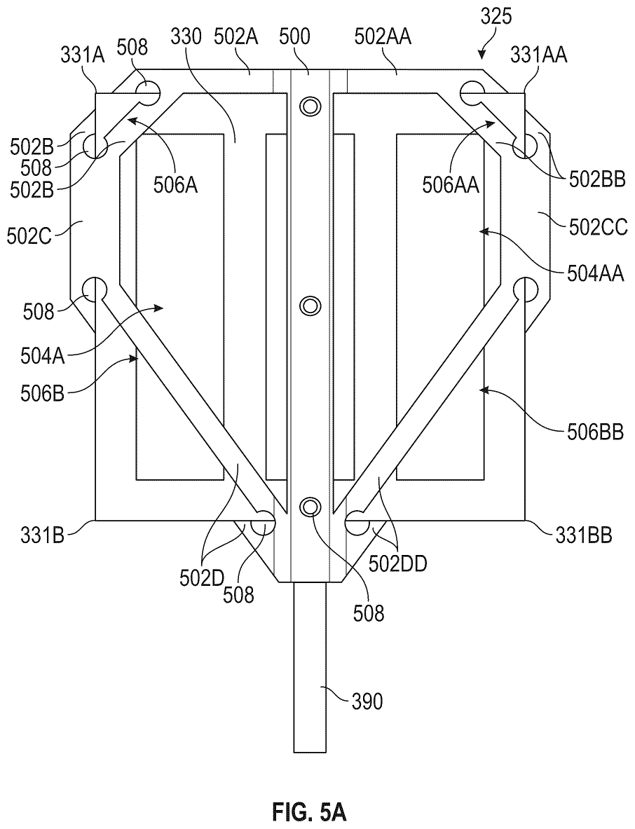

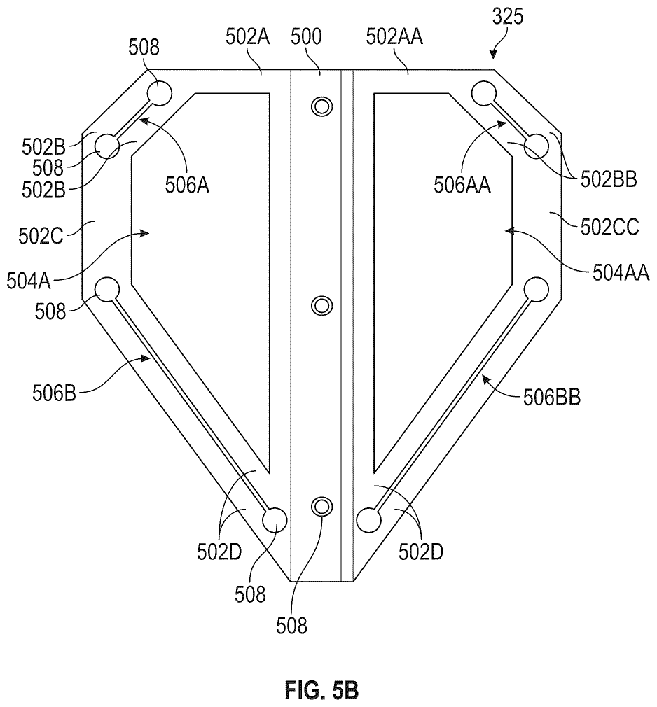

FIG. 4 is an enlarged schematic view of the implant holder 325 of the implant delivery device 300 holding the implant 330. In particular, as shown in FIG. 4, the implant delivery device 300 includes a recess 450 configured to receive implant 330 and a second shaft 430 moveably disposed within recess 450 and configured to support/engage the implant 330 on a second major side thereof. The implant holder 325 can be configured as a card (a relatively thin but wider and longer formed component having features therein) for supporting the implant 330 as further described in reference to FIGS. 5A-8. The implant holder 325 can be fixedly attached to the first shaft 390 at a central hub. The second shaft 430 can be configured to be moveable (e.g., slidable between a first, distal position and a second, proximal position) relative to the first shaft 390. The second shaft 430 can be at least partially received in the first shaft 390 via a cannula of the first shaft 390. In the first, distal position, the second shaft 430 is disposed within recess 450 and engages and secures the implant 330 against the implant holder 325, thus holding the implant 330 in the recess 450 in an unfurled configuration, as shown in FIG. 4. It will be noted that, the implant holder 325 can be configured to be substantially as large as or larger than the implant 330 in some dimensions including in a longitudinal dimension and a lateral (width) dimension. This sizing helps to maintain the implant 330 in a substantially unfurled condition. Depending on the type of implant with which the device is to be used, and the relative stiffness of the implant, the implant holder 325 may vary in stiffness and/or size. In addition, the distance between the implant holder 325 and the second shaft 430 may vary depending on the thickness of the implant and the relative compressibility of the implant in order to ensure suitable clamping force.

As discussed above, the implant holder 325 can be formed of a flexible but resilient material(s) (e.g., a biocompatible thermoplastic polyester elastomer such as Hytrel®). This material(s) may be able to flex relatively easily such as for passage through cannula, but return to the substantially flat configuration. In some embodiments, implant holder 325 formed of such material(s) may have thicknesses of approximately 0.0254 mm to about 0.635 mm. However, other thicknesses depending on the construction of the implant 330 are contemplated. A width in the lateral direction (e.g., transverse to the longitudinal length of the second shaft 430 and the first shaft 390) can be between 25 mm and 35 mm. However, other widths depending on the dimensions of the implant 330 are contemplated. Other possible materials for the implant holder 325 are contemplated and include other biocompatible elastomers, nitinol or the like.

As also shown in FIG. 4, the second shaft 430, which is an inner shaft, can be configured to be translated in the proximal and distal directions. Further, the distal end 315 of implant delivery device 300 may include a recess 445 at the end of first shaft 390 for receiving a distal tip of second shaft 430. Since at least a portion of second shaft 430 is exposed to the surgical site, second shaft 430 may also be formed of a biocompatible material, such as surgical stainless steel or titanium. A distal wall 455 of the recess 450 may be beveled. This bevel may facilitate release of the implant 330, and removal of the implant delivery device 300 from the surgical site as it is withdrawn across a delivered implant. That is, when removing the implant delivery device 300, the bevel of distal wall 455 may enable first shaft 390 to slide past implant 330 without catching on an edge of implant 330.

FIGS. 5A and 5B illustrate the implant holder 325 coupled to a distal part of the first shaft 390. FIG. 5A shows the implant holder 325 retaining the implant 330. FIG. 5B shows the implant holder 325 with the implant removed. FIG. 5A shows the implant 330 retained by the implant holder 325 in a delivery position (e.g., a substantially flat position).

As shown in FIGS. 5A and 5B, the implant holder 325 includes a central hub 500, a plurality of outer edge portions 502A, 502AA, 502B, 502BB, 502C, 502CC, 502D and 502DD, a plurality of windows 504A and 504AA, a plurality of slots 506A, 506AA, 506B and 506BB, and holes 508.

As shown in FIG. 5A, the implant holder 325 can be coupled to the first shaft 390 at the central hub 500. This can be facilitated by some of holes 508 along the central hub 500 being received in projections of the first shaft 390, for example. However, other known mechanical coupling techniques are contemplated. The implant holder 325 can be configured to retain the sheet-like implant 330 adjacent two or more corners 331A, 331AA, 331B and 331BB of the sheet-like implant 330 during implantation of the sheet-like implant 330 into a patient at a surgical site. The two or more corners can include at least the first distal corner 331A and the second distal corner 331AA of the sheet-like implant 330, for example.

The plurality of outer edge portions 502A, 502AA, 502B, 502BB, 502C, 502CC, 502D and 502DD can additionally engage and support the implant 330 in the captured and retained position shown in FIG. 5A. Now referencing both FIGS. 5A and 5B, the outer edge portions 502A, 502AA, 502B, 502BB, 502C, 502CC, 502D and 502DD can be symmetrically arranged about the central hub 500. The outer edge portions 502A, 502AA can form distal edges of the implant holder 325. The outer edge portions 502A, 502AA can connect to and can extend generally laterally outward from the central hub 500. As shown in FIG. 5A, a distal periphery of the implant 330 can be positioned adjacent or at the outer edge portions 502A, 502AA. According to some examples, the outer edge portions 502A, 502AA can extend distal of the distal periphery of the implant 330. The distal periphery of the implant 330 can be engaged by and can be supported by the outer edge portions 502A, 502AA according to some examples.

The outer edge portions 502B, 502BB can connect with the outer edge portions 502A, 502AA. The outer edge portions 502B, 502BB can extend both proximally and laterally outward from the outer edge portions 502A, 502AA to connect with the outer edge portions 502C, 502CC. The outer edge portions 502B, 502BB can form the slots 506A, 506AA, respectively, and can also form some of the holes 508. An area adjacent the first distal corner 331A and the second distal corner 331AA of the implant 330 can be engaged by and be supported by the outer edge portions 502B, 502BB, respectively. Thus, the outer edge portions 502B, 502BB (and indeed others of the plurality of outer edge portions 502A, 502AA, 502B, 502BB, 502C, 502CC, 502D and 502DD) of the implant holder 325 can be configured to retain the sheet-like implant 330 adjacent (within 0.25 mm to about 3.5 mm) the two or more corners (e.g., the first distal corner 331A and the second distal corner 331AA).

The outer edge portions 502C, 502CC can connect with the outer edge portions 502B, 502BB. The outer edge portions 502C, 502CC can extend generally proximally from the outer edge portions 502B, 502BB to connect with the outer edge portions 502D, 502DD. As shown in FIG. 5A, a lateral (width dimension) periphery of the implant 330 can be positioned adjacent or at the outer edge portions 502C, 502CC. According to some examples, the outer edge portions 502C, 502CC can extend further lateral (i.e. can have greater width) than the lateral periphery of the implant 330.

The outer edge portions 502D, 502DD can connect with the outer edge portions 502C, 502CC. The outer edge portions 502D, 502DD can extend both proximally and laterally inward from the outer edge portions 502C, 502CC to connect with the central hub 500. The outer edge portions 502D, 502DD can form the slots 506B, 506BB, respectively, and can also form some of the holes 508. An area spaced a distance inward from the first distal corner 331B and the second distal corner 331BB of the implant 330 can be engaged by and can be supported by the outer edge portions 502D, 502DD.

The plurality of outer edge portions 502A, 502AA, 502B, 502BB, 502C, 502CC, 502D and 502DD can define at least parts of the plurality of windows 504A, 504AA. Further parts of the windows 504A, 504AA can be defined by the central hub 500, for example. In the illustrated example of FIG. 5B, the plurality of windows 504A, 504AA can include two windows including a first window 504A adjacent to and inward of the first distal corner 331A and a second window 504AA adjacent to and inward of the second distal corner 331AA. However, other numbers of windows with different shapes are contemplated. As best shown in FIG. 5A, the plurality of windows 504A and 504AA can be shaped to expose portions of the sheet-like implant 330 for implantation of one or more suture anchors into the sheet-like implant 330.

The plurality of edge portions 502B, 502BB, 502D and 502DD can form the plurality of slots 506A, 506AA, 506B and 506BB, respectively. The implant holder 325 includes holes 508 at an end of each of the plurality of slots 506A, 506AA, 506B and 506BB. The plurality of slots 506A, 506AA, 506B and 506BB can be configured to receive portions of the sheet-like implant 330 therein. The implant holder 325 can be configured to apply a pinch force (e.g., a first compressive force to a first major side of the sheet-like implant and second compressive force to a second major side) to the sheet-like implant during capture of the sheet-like implant with the plurality of slots 506A, 506AA, 506B and 506BB. This pinch force retains the sheet-like implant 330 with application of the generally opposing first compressive force and the second compressive force. The amount of pinch force can be determined by a thickness of the implant holder 325, the material of the implant holder 325, the length and/or orientation of the slots 506A, 506AA and the diameter of the holes 508, for example. According to one example, the diameter of the holes 508 at the edge portions 502B, 502BB, 502D and 502DD can between about 0.5 mm and about 1.5 mm, inclusive. The slots 506A, 506AA, 506B, 506BB can be configured (e.g., sized and oriented) to facilitate disengagement of the sheet-like implant from the implant holder upon withdrawal of the implant holder generally proximally from the surgical site. Furthermore, the pinch force applied by the implant holder 325 can be sufficiently low such that once one or two suture anchors are inserted into the target tissue through the sheet-like implant 330, the implant holder 325 can be disengaged from the sheet-like implant 330 with generally proximal movement of the implant holder 325 without tearing or otherwise damaging the sheet-like implant 330.

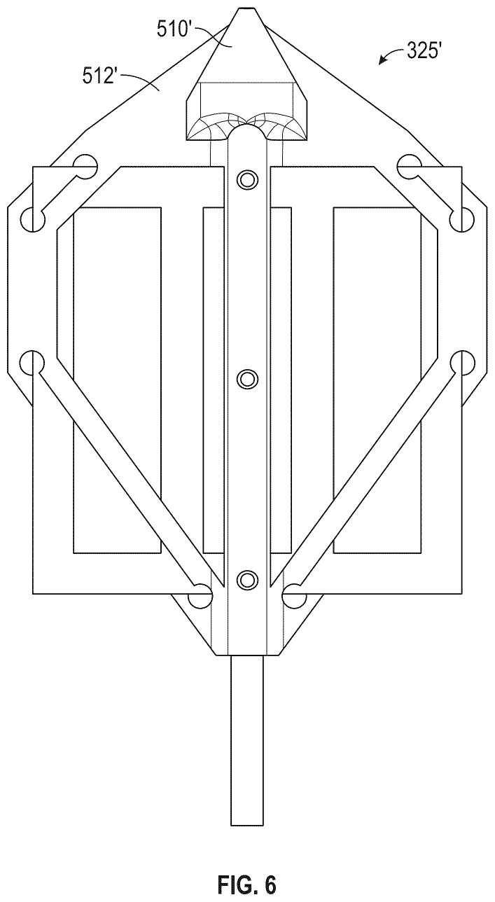

FIG. 6 shows another example of an implant holder 325′ having a construction similar to that of the implant holder 325 discussed previously. The implant holder 325′ differs therefrom as the implant holder 325′ has a bullet 510′ and a modified distal end portion 512′.

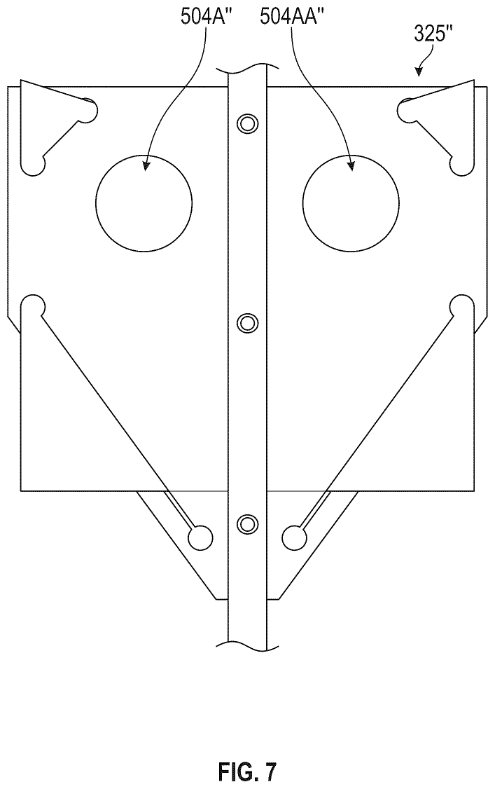

FIG. 7 shows yet another example of an implant holder 325″. The implant holder 325″ differs from the implant holders 325 and 325′ in that windows 504A″ and 504AA″ are smaller. Additionally, the implant holder 325″ can be constructed from multiple layers of card or sheet-like material rather than being constructed from a single layer.

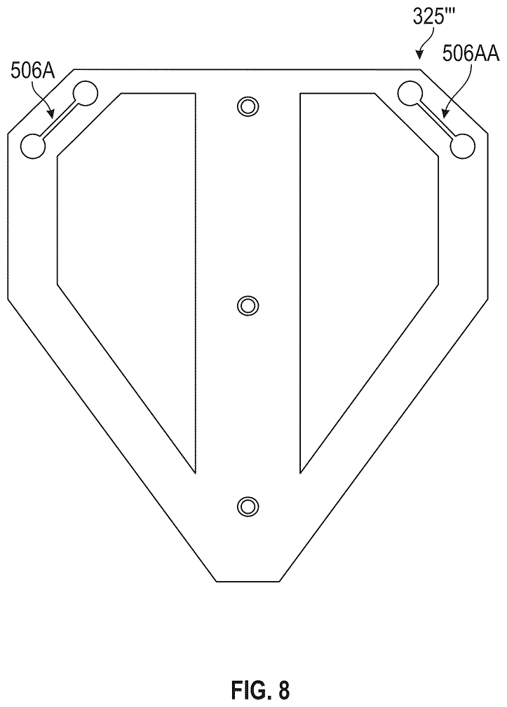

FIG. 8 shows yet another example of an implant holder 325″′. The implant holder 325″′ can have a construction similar to that of the implant holder 325 discussed previously. The implant holder 325′″ differs therefrom as the implant holder 325″′ has only the slots 506A, 506AA adjacent the first distal and the second distal corners thereof. The implant holder 325″′ does not include proximal slots.

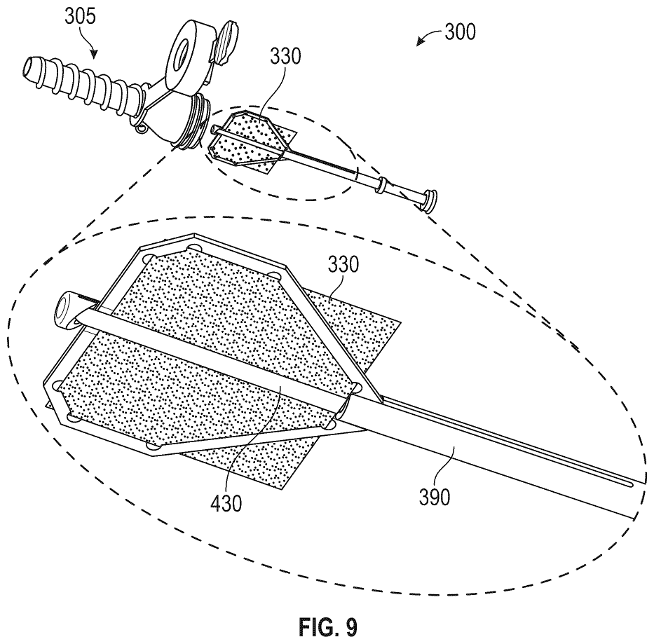

FIG. 9 is another schematic view of implant delivery device 300 holding the sheet-like implant 330 prior to delivery through cannula 305. FIG. 9 also includes an enlarged view of the implant holder 325 of implant delivery device 300. In this view, FIG. 9 shows movable second shaft 430 disposed against implant 330, engaging the implant 330 and preventing implant 330 from being released. FIG. 9 also shows second shaft 430 extending through first shaft 390 and captured in a tip capture feature at the distal end of implant delivery device 300. The tip capture feature can be part of the first shaft 390, for example.

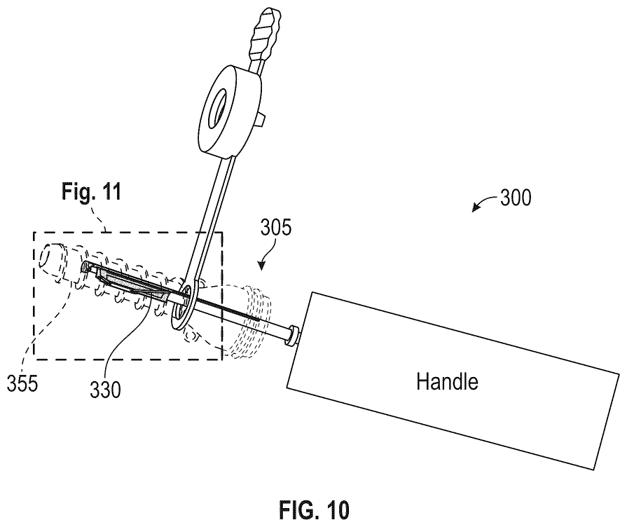

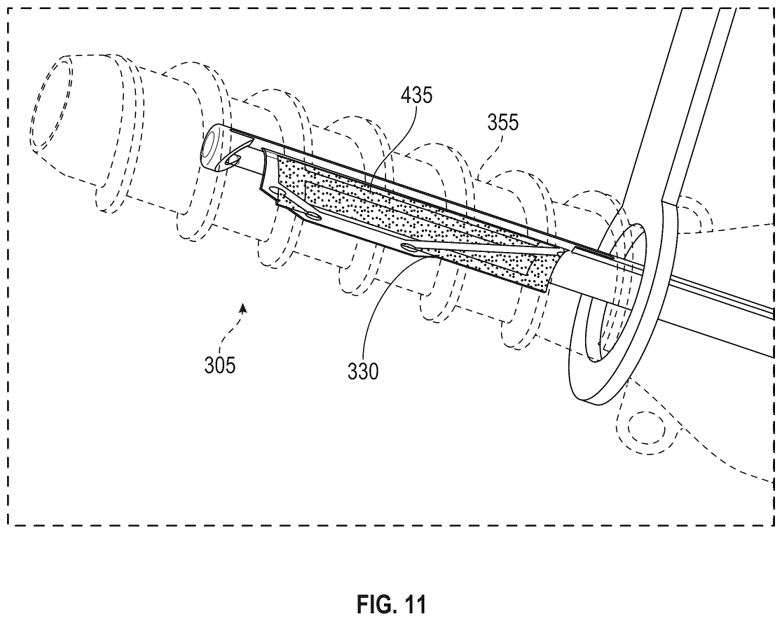

FIGS. 10-11 are schematic views showing the implant 330 being inserted through the cannula 305. FIG. 10 shows cannula 305 in phantom in order to reveal the implant holder 325 of implant delivery device 300 along with implant 330 being passed through cannula 305. As shown in FIG. 10, and more clearly shown in FIG. 11, the sheet-like implant 330 may roll up when being passed through cannula 305.

It will be understood that the implant holder 325 can be flexible and resilient such that the implant holder 325 may be collapsed/deflected upon delivery through cannula 305. In some cases, as shown in FIGS. 10 and 11, the implant holder 325 and implant 330 may roll up in order to facilitate passage of implant 330 through cannula 305 and to provide protection for implant 330 during this passage. It will be understood, however, that the implant and the implant holder may fold, wrinkle, or otherwise collapse in a uniform or non-uniform manner upon insertion through the cannula. In other words, the implant and the implant holder may have a constrained condition or form in which these elements are at least partially collapsed, and an unconstrained condition or form in which these elements are substantially unfurled. The collapsed/constrained condition may occur in situ, as the surgeon passes the distal end of the implant delivery device 300 with the implant 330 through a cannula. This collapsed/constrained condition may be a “non-use” condition of the implant, whereas the (substantially) unfurled condition may be a “use” or delivery condition.

FIG. 12 is a schematic view of the implant delivery device 300 extending through the cannula with the cannula sealing member 400 seated within the cannula 305 and with the implant unfurled to a delivery position (e.g., a substantially flat position) after exiting the cannula 305. In particular, FIG. 12 shows implant holder 325 of implant delivery device 300 extending beyond the distal end of cannula 305. Accordingly, sheet-like implant 330 is unfurled in this delivery position, since the cannula 305 is no longer surrounding implant 330 to keep it rolled up. FIG. 12 also shows cannula sealing member 400 seated within cannula 305. In this configuration of the components, pressure may be maintained within the surgical site and the sheet-like implant 330 is ready to be anchored to the tissue/bone and then released from implant holder 325 of implant delivery device 300.

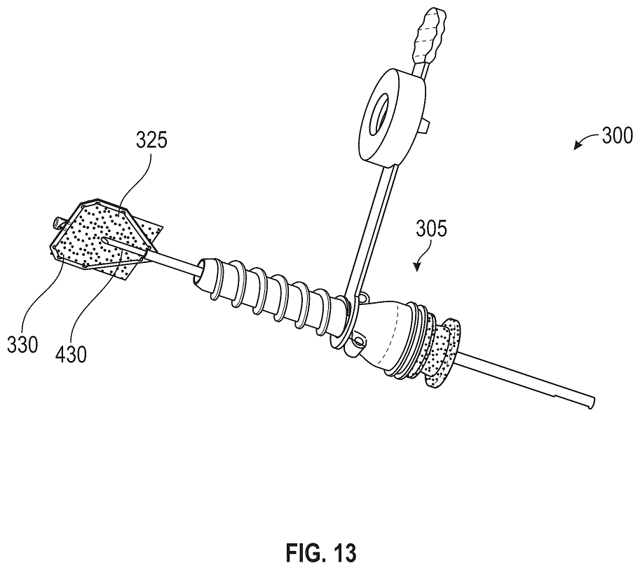

FIG. 13 is a schematic view of the implant delivery device 300 with the second shaft 430 partially withdrawn part way through the process of releasing the sheet-like implant 330.

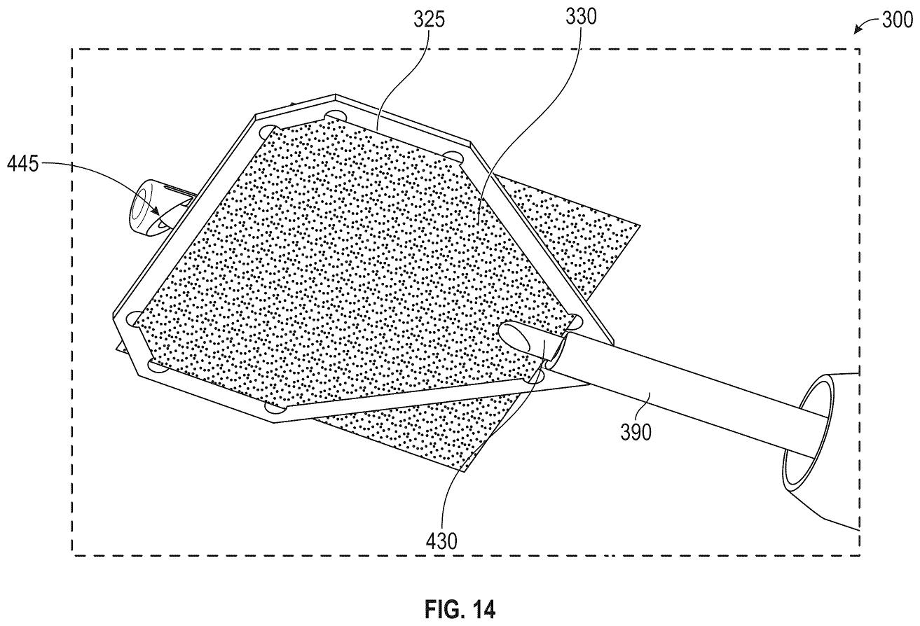

FIG. 14 is an enlarged schematic view of the implant delivery device 300 with the second shaft 430 fully withdrawn. In this condition, the implant 330 is no longer supported on the second major side (the side shown in FIG. 14). FIG. 14 illustrates how the sheet-like implant 330 is supported by the implant holder 325 remains in place, engaging the implant 330 and coupling the implant 330 to first shaft 390.

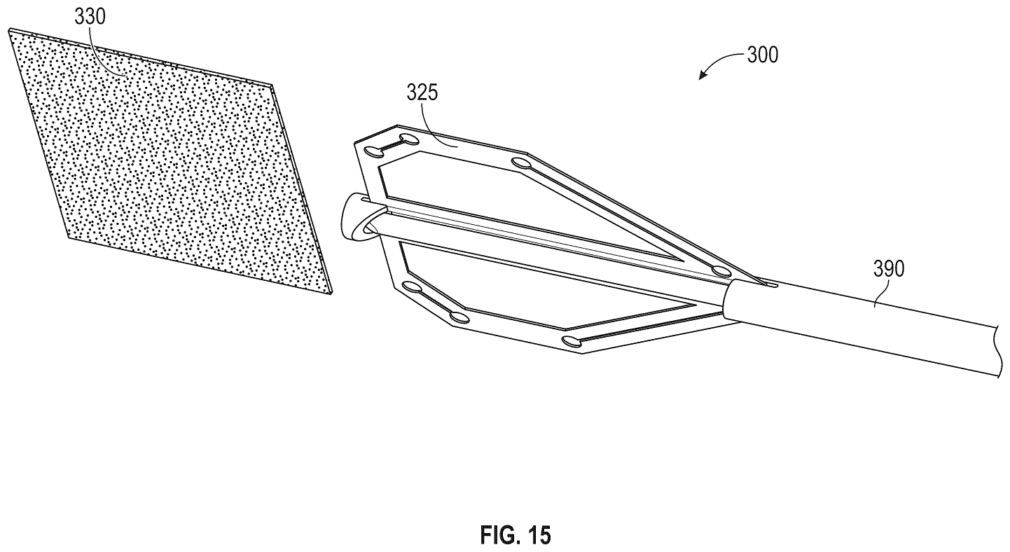

In order to fully release the sheet-like implant 330 from the implant holder 325, the second shaft (not shown) is retracted as previously shown. Additionally, as shown in FIG. 15 suture anchors are applied through the sheet-like implant 330 coupling the sheet-like implant 330 to tissue (not shown). As further shown in FIG. 15, the implant holder 325 and the implant delivery device 300 are withdrawn generally proximally this movement along with the anchoring of the implant 330 causes the implant 330 to be released from the implant holder 325. A shown in FIG. 15, the implant delivery device 300 has been moved further proximally and no longer overlaps implant 330. Accordingly, implant 330 is released from the implant holder 325 and the first shaft 390 of implant delivery device 300. Once implant 330 is secured at the surgical site and released, implant delivery device 300 may be removed from the surgical site through cannula 305.

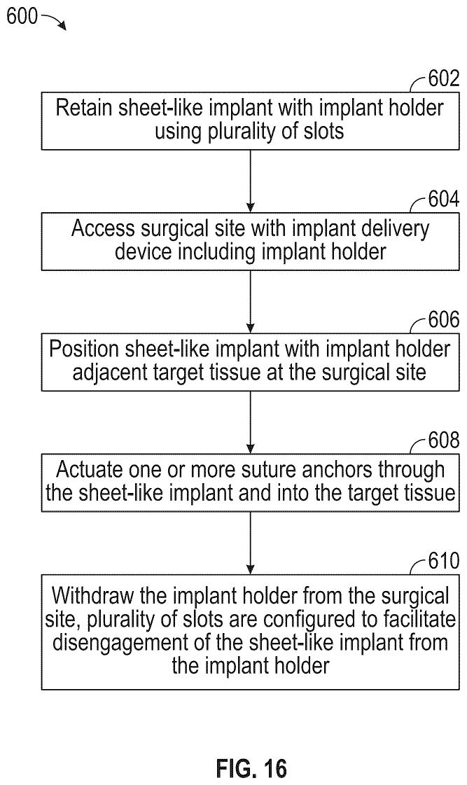

FIG. 16 shows a flow chart of a method 600 of deploying a sheet- like implant into a patient at a surgical site. The method 600 can include retaining 602 the sheet-like implant with an implant holder of an implant delivery device using a plurality of slots which receive the sheet-like implant therein adjacent at least a first distal corner and a second distal corner of the sheet-like implant. The method 600 can include accessing 604 the surgical site with a distal end of the implant delivery device including the implant holder. The method 600 can include positioning 606 the sheet-like implant adjacent a target tissue of the patient at the surgical site. The method 600 can include actuating 608 one or more suture anchors through the sheet-like implant and into the target tissue. The method 600 can include withdrawing 610 the implant holder generally proximally from the surgical site, wherein the plurality of slots are configured to facilitate disengagement of the sheet-like implant from the implant holder upon the withdrawing.

Optionally, the method 600 can optionally include retracting a shaft to disengage the shaft from retaining the sheet-like implant prior to the withdrawing the implant holder generally proximally from the surgical site. The method 600 can include wherein actuating the one or more suture anchors through the sheet-like implant and into the target tissue includes passing the suture anchors through one or more windows of the implant holder. The method 600 can include wherein accessing the surgical site with the distal end of the implant delivery device including the implant holder includes temporarily deforming the implant holder to pass through a cannula. The method 600 can include wherein positioning the sheet-like implant with the implant holder adjacent the target tissue of the patient at the surgical site includes maintaining the sheet-like implant in a substantially flat position adjacent the target tissue of the patient. The method 600 can include wherein the implant holder is configured to pinch a first major side and a second major side of the sheet-like implant during the retaining the sheet-like implant with the implant holder using the plurality of slots.

The foregoing systems, methods and devices are merely illustrative of the components, interconnections, communications, functions, etc. that can be employed in carrying out examples in accordance with this disclosure. Different types and combinations of devices, and other systems and devices can be employed in examples according to this disclosure.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided.

Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein. In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure.

This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

While various embodiments are described, the description is intended to be exemplary, rather than limiting and it will be apparent to those of ordinary skill in the art that many more embodiments and implementations are possible that are within the scope of the disclosed embodiments. Although many possible combinations of features are shown in the accompanying figures and discussed in this detailed description, many other combinations of the disclosed features are possible. Any feature or element of any embodiment may be used in combination with or substituted for any other feature or element in any other embodiment unless specifically restricted. Further, unless otherwise specified, any step in a method or function of a system may take place in any relative order in relation to any other step described herein.

### EXAMPLES

The following, non-limiting examples, detail certain aspects of the present subject matter to solve the challenges and provide the benefits discussed herein, among others.

Example 1 is an implant delivery device, optionally comprising: a handle disposed at a proximal end of the implant delivery device and configured to be grasped by a user; a first shaft extending distally from the handle; and an implant holder coupled to the first shaft, the implant holder being configured to retain a sheet-like implant adjacent two or more corners of the sheet-like implant during implantation of the sheet-like implant into a patient at a surgical site.

In Example 2, the subject matter of Example 1 optionally includes, a second shaft actuated by an actuator of the handle to be moveable along a cannula of the first shaft, wherein the second shaft is selectively engageable with and disengageable from the sheet-like implant.

In Example 3, the subject matter of Examples 1-2 optionally includes, wherein the implant holder includes a plurality of windows exposing portions of the sheet-like implant for implantation of suture anchors into the sheet- like implant.

In Example 4, the subject matter of Example 3 optionally includes, wherein the plurality of windows comprise at least two windows including a first window adjacent to and inward of a first of the two or more corners and a second window adjacent to and inward of a second of the two or more corners.

In Example 5, the subject matter of Examples 3-4 optionally includes, wherein the two or more corners include at least a first distal corner and a second distal corner of the sheet-like implant.

In Example 6, the subject matter of Examples 3-5 optionally includes, wherein the implant holder is configured as a sheet-like deformable card along portions thereof.

In Example 7, the subject matter of Example 6 optionally includes, wherein the portions include a plurality of outer edge portions of the card that define at least parts of the plurality of windows and the portions of the implant holder being configured to retain the sheet-like implant adjacent the two or more corners.

In Example 8, the subject matter of Examples 1-7 optionally includes, wherein the implant holder is made of thermoplastic polyester elastomer.

In Example 9, the subject matter of Examples 1-8 optionally includes, wherein the implant holder is configured to capture the sheet-like implant with a plurality of slots which receive the sheet-like implant therein adjacent the two or more corners of the sheet-like implant.

In Example 10, the subject matter of Example 9 optionally includes, wherein the implant holder is configured to pinch a first major side and a second major side of the sheet-like implant during capture of the sheet-like implant with the plurality of slots.

In Example 11, the subject matter of Example 10 optionally includes, wherein the plurality of slots are configured to facilitate disengagement of the sheet-like implant from the implant holder upon withdrawal of the implant holder generally proximally from the surgical site.

In Example 12, the subject matter of Examples 9-11 optionally includes, wherein the implant holder includes holes at an end of each of the plurality of slots.

In Example 13, the subject matter of Examples 1-12 optionally includes, wherein the implant holder is configured to maintain the sheet-like implant in a substantially flat position adjacent a target tissue of the patient at the surgical site.

Example 14 is an implant delivery device, optionally comprising: a handle disposed at a proximal end of the implant delivery device and configured to be grasped by a user; a first shaft extending distally from the handle; and an implant holder coupled to the first shaft, the implant holder being configured to retain a sheet-like implant using a plurality of slots which receive the sheet-like implant therein adjacent at least a first distal corner and a second distal corner of the sheet-like implant during implantation of the sheet-like implant into a patient at a surgical site.

In Example 15, the subject matter of Example 14 optionally includes, wherein the implant holder is configured to pinch a first major side and a second major side of the sheet-like implant to retain the sheet-like implant with the plurality of slots.

In Example 16, the subject matter of Examples 14-15 optionally includes, wherein the plurality of slots are configured to facilitate disengagement of the sheet-like implant from the implant holder upon withdrawal of the implant holder generally proximally from the surgical site.

In Example 17, the subject matter of Examples 14-16 optionally includes, wherein the implant holder includes holes at an end of each of the plurality of slots.

In Example 18, the subject matter of Examples 14-17 optionally includes, wherein the implant holder is configured to maintain the sheet-like implant in a substantially flat position adjacent a target tissue of the patient at the surgical site.

In Example 19, the subject matter of Examples 14-18 optionally includes, a second shaft actuated by an actuator of the handle to be moveable along a cannula of the first shaft, wherein the second shaft is selectively engageable with and disengageable from the sheet-like implant.

In Example 20, the subject matter of Examples 14-19 optionally includes, wherein the implant holder includes a plurality of windows exposing portions of the sheet-like implant for implantation of suture anchors into the sheet-like implant.

In Example 21, the subject matter of Example 20 optionally includes, wherein the plurality of windows comprise at least two windows including a first window adjacent to and inward of the first distal corner and a second window adjacent to and inward of the second distal corner.

In Example 22, the subject matter of Example 21 optionally includes, wherein the implant holder is configured as a sheet-like deformable card along portions thereof.

In Example 23, the subject matter of Example 22 optionally includes, wherein the portions include a plurality of outer edge portions of the card that define at least parts of the plurality of windows and the portions of the implant holder being configured to retain the sheet-like implant adjacent the first distal corner and the second distal corner.

In Example 24, the subject matter of Examples 14-23 optionally includes, wherein the implant holder is made of thermoplastic polyester elastomer.

Example 25 is a method of deploying a sheet-like implant into a patient at a surgical site, optionally comprising: retaining the sheet-like implant with an implant holder of an implant delivery device using a plurality of slots which receive the sheet-like implant therein adjacent at least a first distal corner and a second distal corner of the sheet-like implant; accessing the surgical site with a distal end of the implant delivery device including the implant holder; positioning the sheet-like implant adjacent a target tissue of the patient at the surgical site; actuating one or more suture anchors through the sheet-like implant and into the target tissue; and withdrawing the implant holder generally proximally from the surgical site, wherein the plurality of slots are configured to facilitate disengagement of the sheet-like implant from the implant holder upon the withdrawing.

In Example 26, the subject matter of Example 25 optionally includes, retracting a shaft to disengage the shaft from retaining the sheet-like implant prior to the withdrawing the implant holder generally proximally from the surgical site.

In Example 27, the subject matter of Examples 25-26 optionally includes, wherein actuating the one or more suture anchors through the sheet-like implant and into the target tissue includes passing the suture anchors through one or more windows of the implant holder.

In Example 28, the subject matter of Examples 25-27 optionally includes, wherein accessing the surgical site with the distal end of the implant delivery device including the implant holder includes temporarily deforming the implant holder to pass through a cannula.

In Example 29, the subject matter of Examples 25-28 optionally includes, wherein positioning the sheet-like implant with the implant holder adjacent the target tissue of the patient at the surgical site includes maintaining the sheet-like implant in a substantially flat position adjacent the target tissue of the patient.

In Example 30, the subject matter of Examples 25-29 optionally includes, wherein the implant holder is configured to pinch a first major side and a second major side of the sheet-like implant during the retaining the sheet-like implant with the implant holder using the plurality of slots.

Example 31 is at least one machine-readable medium including instructions that, when executed by processing circuitry, cause the processing circuitry to perform operations to implement of any of Examples 1-30.

Example 32 is an apparatus comprising means to implement of any of Examples 1-30.

Example 33 is a system to implement of any of Examples 1-30.

Example 34 is a method to implement of any of Examples 1-30.

Example 35 is any combination of elements or features of Examples 1-30.

What is claimed is:

1. An implant delivery device, comprising:
a handle disposed at a proximal end of the implant delivery device and configured to be grasped by a user;
a first shaft extending distally from the handle; and
an implant holder coupled to the first shaft, the implant holder being configured to retain a sheet-like implant adjacent two or more corners of the sheet-like implant during implantation of the sheet-like implant into a patient at a surgical site.

2. The implant delivery device of claim 1, further comprising a second shaft actuated by an actuator of the handle to be moveable along a cannula of the first shaft, wherein the second shaft is selectively engageable with and disengageable from the sheet-like implant.

3. The implant delivery device of claim 1, wherein the implant holder includes a plurality of windows exposing portions of the sheet-like implant for implantation of suture anchors into the sheet-like implant.

4. The implant delivery device of claim 3, wherein the plurality of windows comprise at least two windows including a first window adjacent to and inward of a first of the two or more corners and a second window adjacent to and inward of a second of the two or more corners.

5. The implant delivery device of claim 4, wherein the two or more corners include at least a first distal corner and a second distal corner of the sheet-like implant.

6. The implant delivery device of claim 5, wherein the implant holder is configured as a sheet-like deformable card along portions thereof.

7. The implant delivery device of claim 6, wherein the portions include a plurality of outer edge portions of the card that define at least parts of the plurality of windows and the portions of the implant holder being configured to retain the sheet-like implant adjacent the two or more corners.

8. The implant delivery device of claim 1, wherein the implant holder is made of thermoplastic polyester elastomer.

9. The implant delivery device of claim 1, wherein the implant holder is configured to capture the sheet-like implant with a plurality of slots which receive the sheet-like implant therein adjacent the two or more corners of the sheet-like implant.

10. The implant delivery device of claim 9, wherein the implant holder is configured to pinch a first major side and a second major side of the sheet-like implant during capture of the sheet-like implant with the plurality of slots.

11. The implant delivery device of claim 10, wherein the plurality of slots are configured to facilitate disengagement of the sheet-like implant from the implant holder upon withdrawal of the implant holder generally proximally from the surgical site.

12. The implant delivery device of claim 9, wherein the implant holder includes holes at an end of each of the plurality of slots.

13. The implant delivery device of claim 1, wherein the implant holder is configured to maintain the sheet-like implant in a substantially flat position adjacent a target tissue of the patient at the surgical site.

14. An implant delivery device, comprising:
a handle disposed at a proximal end of the implant delivery device and configured to be grasped by a user;
a first shaft extending distally from the handle; and
an implant holder coupled to the first shaft, the implant holder being configured to retain a sheet-like implant using a plurality of slots which receive the sheet-like implant therein adjacent at least a first distal corner and a second distal corner of the sheet-like implant during implantation of the sheet-like implant into a patient at a surgical site.

15. The implant delivery device of claim 14, wherein the implant holder is configured to pinch a first major side and a second major side of the sheet-like implant to retain the sheet-like implant with the plurality of slots.

16. The implant delivery device of claim 14, wherein the plurality of slots are configured to facilitate disengagement of the sheet-like implant from the implant holder upon withdrawal of the implant holder generally proximally from the surgical site.

17. The implant delivery device of claim 14, wherein the implant holder includes holes at an end of each of the plurality of slots.

18. The implant delivery device of claim 14, wherein the implant holder is configured to maintain the sheet-like implant in a substantially flat position adjacent a target tissue of the patient at the surgical site.

19. The implant delivery device of claim 14, further comprising a second shaft actuated by an actuator of the handle to be moveable along a cannula of the first shaft, wherein the second shaft is selectively engageable with and disengageable from the sheet-like implant.

20. The implant delivery device of claim 14, wherein the implant holder includes a plurality of windows exposing portions of the sheet-like implant for implantation of suture anchors into the sheet-like implant, wherein the plurality of windows comprise at least two windows including a first window adjacent to and inward of the first distal corner and a second window adjacent to and inward of the second distal corner, wherein the implant holder is configured as a sheet-like deformable card along portions thereof, wherein the portions include a plurality of outer edge portions of the card that define at least parts of the plurality of windows and the portions of the implant holder being configured to retain the sheet-like implant adjacent the first distal corner and the second distal corner.
